(19) United States
(12) Patent Application Publication
(10) Pub. No.: US 2008/0009738 A1
(43) Pub. Date: Jan. 10, 2008

(54) Method for Utilizing User Input for Feature Detection in Diagnostic Imaging

(75) Inventors: Xiang-Ning Li, Mill Creek, WA (US); Paul Detmer, Seattle, WA (US); Antoine Collet-Billon, Paris (FR); Olivier Gerard, Viroflay (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 10/578,978

(22) PCT Filed: Nov. 15, 2004
(86) PCT No.: PCT/IB04/52431

§ 371 Date: May 25, 2007

Related U.S. Application Data
(60) Provisional application No. 60/520,578, filed on Nov. 17, 2003.

(51) Int. Cl. A61B 8/14

(52) U.S. Cl. 600/458; 703/11

(57) ABSTRACT

A method for utilizing user input for segmentation and feature detection in diagnostic ultrasound imaging is provided. The method provides border detection to be performed rapidly; a feature, which allows the border detection method to be applied to real-time video imaging with little or no delay. The border detection method may be incorporated within a diagnostic ultrasound imaging system or as a user-installable software application capable of being installed on and executed by an ultrasound imaging system.

The present invention relates to ultrasound diagnostic imaging. Specifically, the present invention relates to a method for utilizing user input for feature detection in diagnostic imaging.

Ultrasound has become an important tool in medical diagnostics. Ultrasound's non-invasive and generally benign radiation-free imaging has found wide spread use, especially in fetal imaging and extended exposure video imaging. While ultrasound has good penetration through the soft tissues of the human body, there is no way to prevent reflection of overlaying structures from obscuring the areas of interest during the imaging process. This becomes particularly important for 3D or Live 3D ultrasound imaging where the relationship of the overlying structures may have a complex relationship with the viewing planes or volume being observed

Attempts have been made to provide a way to remove obscuring or distracting regions from ultrasound images and volumes. One such method requires the operator to manually select and remove the obscuring region within an ultrasound imaging application. While this method provides adequate accuracy, it is a very time consuming process and thus not appropriate for real-time ultrasound imaging applications.

A second method provides an automatic selection process wherein the operator initially identifies the region of interest, perhaps by selecting from a menu of choices or manually selecting the region, and from that point on the ultrasound imaging software automatically detects and removes or de-emphasizes the obscuring portions of the ultrasound image. This method can be significantly faster and therefore has the potential of being very useful in real-time applications. However, this method too has its drawbacks. While corporeal structures have the same basic shape from one person to another, size may differ, disease may alter the shape of the structure, and even the particular position of the ultrasound transducer during imaging may cause the structure to appear altered from its typically accepted shape. These variations in shape can lead to misidentification of regions by the automatic selection process. This possibility for misidentification also contributes to an operator's reluctance to rely on the automatic selection process, defeating the purpose of supplying such a process in the ultrasound imaging software.

What is needed is a selection process that is both accurate enough to inspire trust from the operator and fast enough to be applicable in real-time imaging applications. An object of the present invention is to provide a method that, with limited input from the operator, is able to identify regions of interest in an ultrasound image or volume, both still and live, and remove or de-emphasize obstructions thereon.

The present invention provides a method for utilizing user input for segmentation and feature detection in diagnostic imaging. By using the detection methodology of the present invention, structures can be identified and either emphasized or de-emphasized based on their position within an operator-specified region of interest.

The method of the present invention for defining internal structural borders in a medical ultrasonic image includes several steps. Initially, an ultrasonic image or volume region having a region of interest is acquired. A feature of interest is located in the ultrasonic image or a plane of the volume and at least one side of the shape is placed in a proximal relationship to the feature. At least one starting point within at least one shape is identified. The starting point is used for detecting and delineating a tissue border within the ultrasonic image or tissue surface within the volume. The tissue border detection is performed using internally stored complex shapes having fuzzy border regions instead of solid linear borders. As more points are located, the border regions may be adjusted to produce a best-fit based on the currently located points. An indicator is provided for identifying and highlighting the tissue border on the ultrasound image to the operator. The indicator may include emphasizing the tissue structure by colorizing or enhancing the contrast of the region bounded by the detected tissue structure.

Additionally, the present invention may allow the operator to interactively modify the shape placed in proximal relationship to a feature. The operator modifies the shape so that it more close matches or approximates the region of interest, i.e. if the region of interest is generally oval or ellipsoidal in shape, then the operator may select a circular shape, place the shape over the region of interest, and deform the circular shape to obtain an oval of approximately similar dimensions as the region of interest.

The foregoing objects and advantages of the present invention may be more readily understood by one skilled in the art with reference being had to the following detailed description of preferred embodiments thereof, taken in conjunction with the accompanying drawings in which:

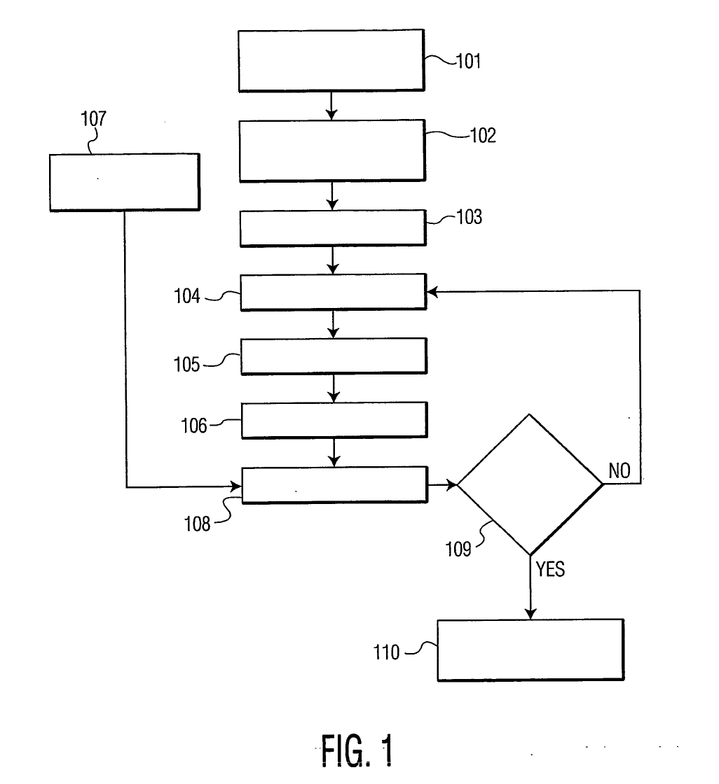

FIG. 1 is a flowchart illustrating the steps performed by the method for utilizing user input for border detection in diagnostic imaging in accordance with the present invention; and

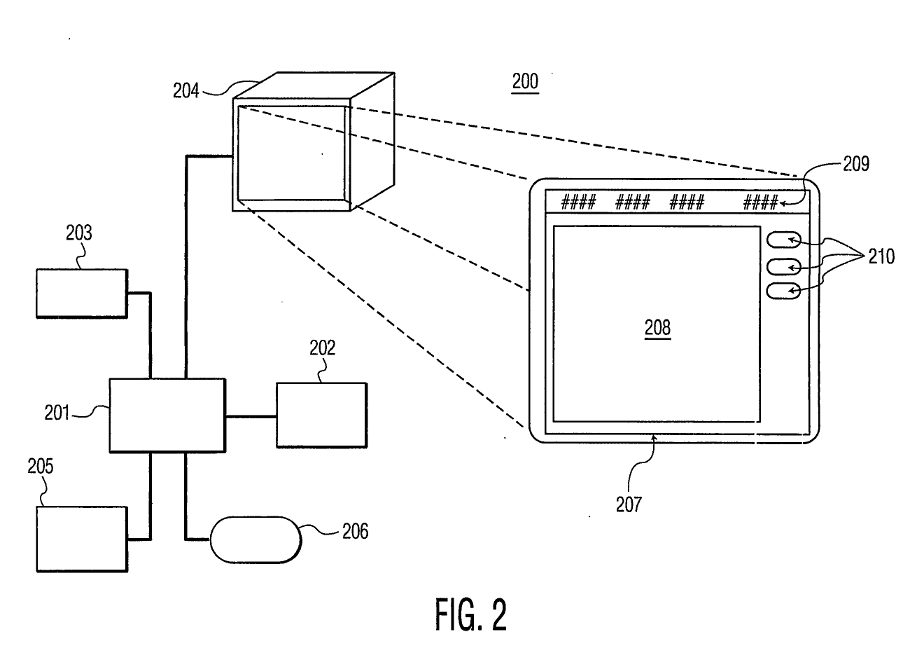

FIG. 2 is a block illustration of a system for border detection in diagnostic imaging in accordance with the present invention.

Several embodiments of the present invention are hereby disclosed in the accompanying description in conjunction with the figures. Preferred embodiments of the present invention will now be described in detail with reference to the figures wherein like reference numerals identify similar or identical elements.

An embodiment of the present invention, shown in FIG. 1, provides a method for defining internal structural borders in a medical ultrasound image. In step 101, an ultrasound imaging system images a patient or other object appropriately capable of being imaged by ultrasound energy. The ultrasound imaging system transfers the ultrasound image(s) or volume data to an electronic data storage device, e.g. volatile and non-volatile memory, magnetic media, optical media, etc. in step 102. The image data is also displayed on a display screen having an interface configured for providing an operator controllable image processing and analysis functionality in step 103.

In step 104, an operator selects one or more region(s) of interest (RoI) on the displayed image data as the desired starting point (seed). The interface allows the operator to indicate the RoI (either in 2D or 3D) by selecting one or more shapes from amongst a variety of simple geometric models, e.g. circle, square, polygon, slice, cube, sphere, etc., and placing the selected model on the image so that the model bounds the RoI. Additionally the interface provides a method for the operator to indicate the ultrasound image type, for example, cardiac, fetal, etc.

The image type and bounded RoI are used by the system for analyzing the area within the region(s) of interest in step 105. Contours and structures within the RoI are detected in step 106. A method for delineating these contours and structures includes adjusting contrast and colorizing structures according to predefined or operator-definable preferences, and display of the resulting image data on the display screen are provided in step 108. The delineation preferences to be applied to the RoI of the ultrasound image are set in step 107 prior to execution of step 108.

In step 109, the operator is given the opportunity to review and either accept the image processing as displayed in step 108 or reject it if the RoI is not acceptably displayed. If the results of step 108 are acceptable, the process is completed. However, if the operator rejects the results of step 108, step 104 is executed again, giving the operator an opportunity to adjust the RoI selection as well as the image type in an attempt to refine the resulting image data in step 108. The subsequent steps are executed as described above.

Additional features may be incorporated into the present embodiment of the invention, for example, the method may include a process by which the system can learn and adapt over time based on the feedback received from the operator in step 109. Further, image processing and manipulation functions may also be provided by the system, such as enlarging, rotating, and cropping the RoI.

The analysis and detection steps are performed by the present embodiment through analytical algorithms, which use predetermined and internally located complex shapes approximating the general shapes of various bodily tissues and structures. The indicated image type is used to identify which of the variety of complex shapes are to be applied to the RoI analysis. However, as discussed previously the imaged shape of a bodily tissue or structure may appear different from-the typically associated shape of the tissues and structure due to various factors such as the angle and position of the ultrasound imaging unit. For this reason, the present embodiment utilizes a fuzzy model of these tissues and structures. In this case fuzzy is meant to indicate that the complex shapes have, as their boundaries, a predefined acceptable range (e.g., maximal size limit) instead of a sharply defined boundary, thus a tissue boundary point need not lie directly on the boundary of the corresponding complex shape but merely within the acceptable range. Additionally, as more of the tissue boundary points are detected on the image, the acceptable range of the boundary of the complex shape may be adjusted as appropriate, “on the fly” or in real-time, based on the location of the detected points. These detections and adjustments are performed automatically by the present embodiment.

An alternate embodiment of the present invention allows the operator to adjust a shape selected from the variety of simple geometric models following the execution of step 104. Thus, the shape used to indicate the RoI can more closely match the actual shape of the region and, consequently, increase the accuracy and speed of the analyzing and detection steps. Additionally, if in step 109, the results from step 108 are rejected, then the originally selected shape of step 104 can be modified to increase the likelihood of a successful end result from step 108.

The method as described above may be implemented as a software application or set of processor commands and installable onto a pre-existing ultrasound diagnostic system. In this embodiment, the software application may be stored on any of variety of commonly used computer readable media, such as compact disc, DVD, and magnetic media, or as a network downloadable software package.

Another embodiment, as shown in FIG. 2, provides an ultrasound diagnostic system 200 configured and disposed for executing the steps of the present invention as described above. The system 200 includes a controller/processor unit 201, having a user input device(s) 202, such as keyboard, mouse, speech recognition device, etc., a storage device 203, and a display screen 204, connected with and configured for controlling an ultrasound imaging device 206, such as an ultrasonic probe. An optional, hard copy output device 205, such as a printer, may also be present and connected to the controller/processor unit 201.

A software application or set of processor commands, residing within the controller/processor unit 201 or stored on the storage device 203, is configured to execute the steps of the method of the present invention as shown in FIG. 1 and described above. The controller/processor unit 201, upon receiving an actuation signal from the operator via the user input device(s) 202, activates the ultrasound imaging device 206. The actuation signal may include or be preceded by a set of operator-adjustable preference signals which are used by the controller/processor unit 201 to adjust the parameters of the ultrasound imaging device 206. The ultrasound imaging device 206 transmits high frequency audio signals toward a patient or object (not shown) to be imaged and receives signals reflected from structures internal to the scanned patient or object in a manner well known in the art. The received signals are transferred to the controller/processor unit 201 for further processing.

The controller/processor unit 201 processes signals and displays a corresponding image 208 on the display screen 204. Additionally, the controller/processor unit 201 provides an interface, preferably a graphical user interface (GUI) 207, which allows the operator to selectively indicate a region of interest (RoI) on the displayed image 208. The interface may consist of any of a combination of interface elements, such as menus 209, buttons 210 and icons (not shown) configured to provide predetermined functions. The operator selects the RoI by selecting one or more shape(s) from a variety of simple geometric shapes—square, slice, circle, cube, sphere, etc.—provided by the interface 207 and positioning the shape(s), orientation and size over the RoI such that the RoI is bounded approximately to the boundaries of the shape(s). Further, the operator indicates the type of ultrasound image being displayed through manipulation of interface elements 209, 210, etc. Based on these few inputs from the operator, the controller/processor unit 201 applies predefined algorithms to the RoI for enhancing the various structures contained within the RoI.

The described embodiments of the present invention are intended to be illustrative rather than restrictive, and are not intended to represent every embodiment of the present invention. Various modifications and variations can be made without departing from the spirit or scope of the invention as set forth in the following claims both literally and in equivalents recognized in law.

1. A method of defining internal structural borders in a medical ultrasonic image (208) comprising the steps of:
placing at least one geometric shape in a proximal relationship to a feature in the ultrasonic image (208);
locating at least one starting point within the at least one geometric shape; and
detecting a tissue border and/or structure within a portion of the ultrasonic image (208) bordered by the at least one shape, the detection is performed using one or more shapes selected from a set of predetermined shapes, each having generally the shape of a bodily tissue or structure and a fuzzy border region.

2. The method of claim 1 further comprising the step of displaying the ultrasonic image (208) with delineations, the delineations identifying the detected tissue border and/or structure.

3. The method of claim 1, wherein the placing step further includes adjusting at least one parameter from a set of parameters of the at least one selected geometric shape to approximate the shape of the feature.

4. The method of claim 3, wherein the set of parameters includes size, position and orientation.

5. An ultrasound imaging system configured and disposed for defining internal structural borders in a medical ultrasonic image (208) comprising:
an ultrasound transducer probe (206) configured for producing ultrasound signals, directing the ultrasound signals towards a target to be imaged, and detecting the ultrasound signals reflected from the target;
a display screen (204) for displaying the reflected ultrasound signals in an operator-viewable format;
means for enabling an operator to indicate a region of interest (RoI) by placing at least one geometric shape in a proximal relationship to the RoI; and
a processor (201) comprising:
means for locating at least one starting point within the at least one shape; and
means for detecting a tissue border and/or structure within a portion of the ultrasonic image (208) bordered by the at least one shape by using one or more shapes selected from a set of predetermined shapes, each having generally the shape of a bodily tissue or structure and a fuzzy border region.

6. The system of claim 5, wherein the display screen (204) displays the ultrasound image (208) with a plurality of delineations, the delineations identifying the tissue border and/or structure on the display screen.

7. The system of claim 5, further comprising means for adjusting at least one parameter from a set of parameters of the at least one selected geometric shape to approximate the shape of the feature.

8. The system of claim 6, wherein the set of parameters includes size, position and orientation.

9. A computer readable medium comprising a set of computer readable instructions capable of being executed by at least one processor (201) for defining internal structural borders in a medical ultrasonic image (208) comprising the steps of:
placing at least one geometric shape in a proximal relationship to a feature in the ultrasonic image (208);
locating at least one starting point within the at least one geometric shape; and
detecting a tissue border and/or structure within a portion of the ultrasonic image (208) bordered by the at least one shape using one or more shapes selected from a set of predetermined shapes, each having generally the shape of a bodily tissue or structure and a fuzzy border region.
